(19) United States
(12) Patent Application Publication
(10) Pub. No.: US 2007/0038279 A1
(43) Pub. Date: Feb. 15, 2007

(54) Electrodes for implantable medical devices

(76) Inventors: Daniel W. Fifer, Windsor, CA (US); Terrance Ransbury, Chapel Hill, NC (US)

Correspondence Address: STALLMAN & POLLOCK LLP, 353 SACRAMENTO STREET, SUITE 2200, SAN FRANCISCO, CA, 94111, US

(21) Appl. No.: 11/504,396

(22) Filed: Aug. 15, 2006

Related U.S. Application Data
(60) Provisional application No. 60/708,187, filed on Aug. 15, 2005.

(51) Int. Cl. A61N 1/00

(52) U.S. Cl. 607/116

(57) ABSTRACT

A device for implantation in the vasculature of a patient can include discrete but electrically connected electrodes that, along with the device body, form a substantially smooth exterior surface that allows for easy insertion and removal. Any interstices between the electrodes can be back-filled with a flexible elastomer such as silicone to ensure a smooth surface. The individual electrode segments, such as ring or disc segments, can be connected by an appropriate conductive connection, such as a flexible u-joint, thru-cable, or end-to-end connection including a coupler spring.

The present application claims priority to U.S. Provisional Application No. 60/708,187, filed Aug. 15, 2005.

### TECHNICAL FIELD OF THE INVENTION

The present invention relates to systems and methods for implanting medical devices into a patient's vasculature, such as to sense electrical activity and/or electrically stimulate the heart.

### BACKGROUND

There are a number of medical devices that can have portions implanted into a patient's vasculature. For example, devices such as pacemakers and implantable cardioverter-defibrillators (ICDs) have been successfully implanted for years for treatment of heart rhythm conditions. Pacemakers are implanted to detect periods of bradycardia and deliver electrical stimuli to increase the heartbeat to an appropriate rate, while ICDs are implanted in patients to cardiovert or defibrillate the heart by delivering electrical current directly to the heart. Another implantable defibrillation device can detect an atrial fibrillation (AF) episode and deliver an electrical shock to the atria to restore electrical coordination.

Next generation ICDs, pacemakers, etc., may take the form of elongated intravascular devices, such as those described, for example, in U.S. Pat. No. 7,082,336, entitled “IMPLANTABLE INTRAVASCULAR DEVICE FOR DEFIBRILLATION AND/OR PACING,”; U.S. patent application Ser. No. 10/453,971, entitled “DEVICE & METHOD FOR RETAINING A MEDICAL DEVICE WITHIN A VESSEL”, filed Jun. 4, 2003; as well as U.S. patent application Ser. No. 10/862,113, entitled “INTRAVASCULAR ELECTROPHYSIOLOGICAL SYSTEM AND METHODS,” filed Jun. 4, 2004, each of which is hereby incorporated herein by reference. Such a device can be implanted in a number of alternative ways, including methods described in U.S. patent application Ser. No. 10/862,113, filed Jun. 4, 2004, incorporated by reference above. For example, the device can be introduced into the venous system via the femoral vein, introduced into the venous system via that subclavian vein or the brachiocephalic veins, or into the arterial system using access through one of the femoral arteries. Moreover, different components of the intravascular systems may be introduced through different access sites. For example, a device may be separately introduced through the femoral vein and a corresponding lead may be introduced via the subclavian vein.

According to embodiments disclosed in the above-referenced applications, a pulse generator may be implanted within a blood vessel, with the pulse generator being proportioned to allow blood flow through the blood vessel. At least one electrode is electrically coupled to the pulse generator, typically positioned about an outer circumference of the device. A lead having another electrode is positioned elsewhere in the cardiovascular system, such as in the right ventricle (RV) or left subclavian vein. An exposed or open coil for a prior art lead such as RV leads used in the prior art can allow in-growth of fibrous tissue, thus compromising or complicating the retrieval of the lead. An example of an open coil 100 about a lead 102 of the prior art is shown in FIG. 1. As can be seen, the coil provides undulations that can promote fibrous ingrowth about the lead, which can lead to damage of the surrounding tissue upon extraction of the lead 102.

### BRIEF DESCRIPTION OF THE DRAWINGS

FIG. 1 is a diagram of a helical electrode of the prior art in accordance with one embodiment of the present invention.

FIG. 2 is a side elevation view of a first embodiment of an electrode assembly.

FIGS. 3A, 3B and 3C are side elevation views of coupling assemblies suitable for use in the electrode assembly of FIG. 2.

FIG. 4 is a side elevation view of an alternative electrode assembly utilizing a bellows-shaped electrode.

FIG. 5 is a side elevation view of an alternative electrode assembly having enhanced electrode surface area.

FIGS. 6A-6C are a sequence of steps illustrating manufacturing of an alternative electrode assembly utilizing a helical electrode.

FIG. 6D is a cross-section view of a wall of the lead of FIG. 6C, illustrating the detail of the region enclosed by circle 6D-6D in FIG. 6C.

FIGS. 7A-7D are longitudinal cross-section views similar to FIG. 6D showing portions of lead walls for alternative electrode assemblies.

FIG. 7E is an elevation view of the electrode of FIG. 7D before application of the elastomeric material.

FIG. 7F is an elevation view of an alternative electrode that may be used in the embodiment of FIG. 7D.

FIG. 8A is a cross-sectional side view illustrating use of a mandrel to form an electrode assembly of the type shown in FIG. 7B.

FIG. 8B is a cross-section view taken along the plane designated 8B-8B in FIG. 8A.

FIG. 9 is a detail view of the region enclosed by circle 9-9 in FIG. 8B.

FIG. 10 is a diagram of a molding assembly that can be used to form an electrode assembly of the type disclosed herein.

FIGS. 11A and 11B illustrate use of the molding assembly of FIG. 10.

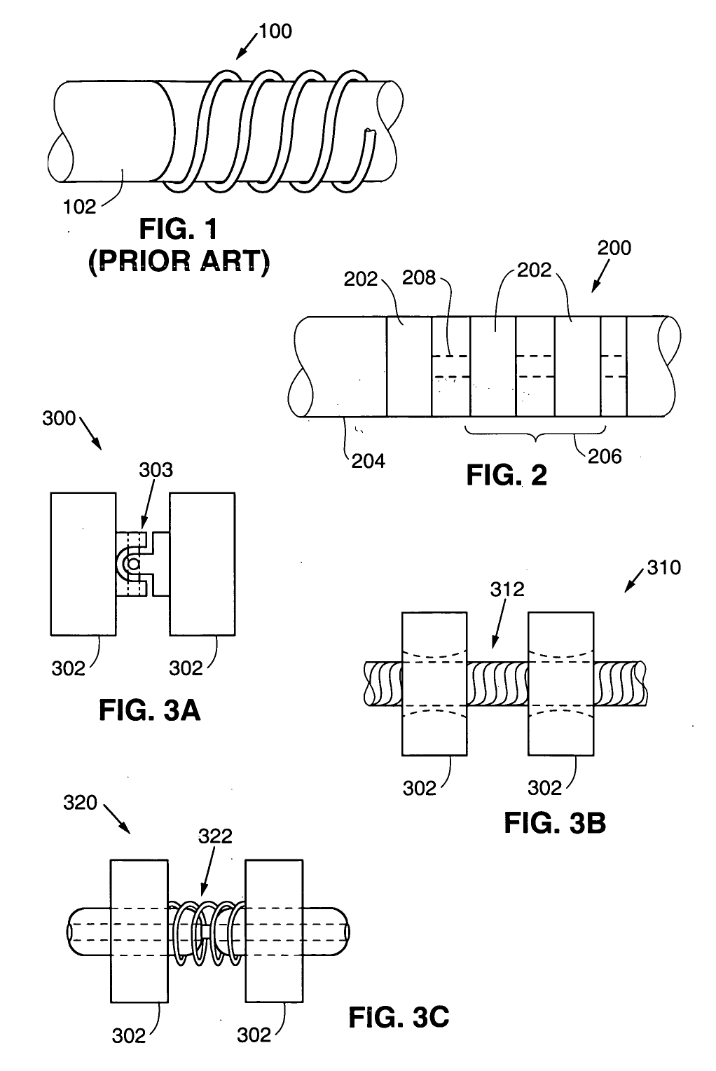

### DETAILED DESCRIPTION

Systems and methods in accordance with various embodiments of the present invention overcome deficiencies in existing implantable devices by including features that can facilitate or at least accommodate device removal by minimizing or preventing damage to surrounding tissue upon lead extraction. The disclosed electrode assemblies are suitable for use on devices (e.g. pulse generators) and/or electrode leads implantable in the heart, vasculature, or elsewhere in the body. In any of the disclosed embodiments, the electrodes can be tapered or coated as necessary to minimize thrombosis etc, and can be formed of any appropriate biocompatible and conductive material. The circuitry and other electronic and physical components necessary to provide power to the electrodes are well known in the art and will not be discussed herein in detail.

Discrete Segments

FIG. 2 shows a lead device 200 including an electrode 202 in accordance with a first embodiment. In this exemplary device, the electrode assembly includes discrete but electrically connected electrodes 202. The lead body 204 and electrodes 202 form a smooth exterior surface 206 that allows for easy insertion and removal of the lead device. Any interstices between the electrodes can be back-filled with a flexible elastomer such as silicone to ensure a smooth surface. The individual electrode segments, such as ring or disc segments, can be connected by an appropriate conductive connection 208. While the diagram shows the electrodes connected via a simple wire, there can be any of a number of ways to connect the electrodes, in order to provide strength and a level of flexibility. FIGS. 3A-3C show a number of exemplary ways to electrically connect the discrete segments. For example, one connection approach 300 shown in FIG. 3A 300 uses a flexible u-joint 302 to connect the electrode segments 302. Another connection approach 310 shown in FIG. 3B utilizes a thru-cable 312 to connect the electrode segments, while still another approach 320 (FIG. 3C) utilizes an end-to-end connection including a coupler spring 322. These connections can be made using any appropriate material, and can provide any desirable amount of strength, flexibility, and/or rigidity. These approaches can be used alone or in combination. The discrete electrode segments can float on the connector(s), but be constrained by the elastomer filling the surrounding spaces and thus held in electrical contact with the associated conductors. Alternatively, the segments also can be mechanically connected to each other and/or to the conductors, such as through a crimp or weld.

Bellows Electrode

An electrode assembly 400 in accordance with a second embodiment is shown in FIG. 4. In this embodiment, the electrode can be constructed from a single (or multiple) bellows 402 shaped to have approximately the same outer diameter as the lead 404, such that a portion of each section 406 will be exposed to the outside of the lead assembly. The bellows can be back filled with a material such as silicone to create a smooth, cylindrical profile while still allowing the sections to be exposed for electrical coupling to the patient. The bellows and backfill material can be made of any appropriate material discussed herein or known in the art. The bellows also can provide a level of flexibility to the lead assembly.

Surface Area Enhancement

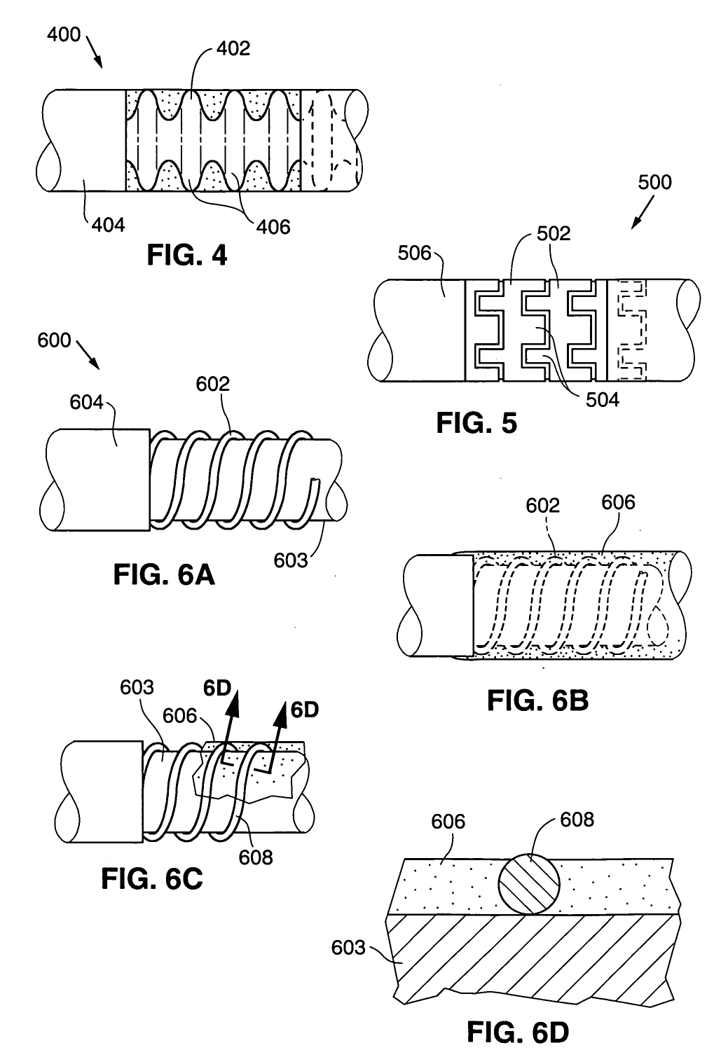

While the bellows arrangement can provide a level of flexibility and simplicity, there may not be enough electrode area exposed to the surface for all applications. As such, the amount of exposed surface area can be increased by using an electrode design 500 in accordance with a third embodiment, such as is shown in FIG. 5. In this embodiment, the electrode surface area is increased by using a non-ring shape geometry. In other words, even though the electrode segments 502 might have a circular outer diameter, and might have a central opening, the front and back sides of each segment are not substantially planar as in a ring segment, but instead have extension portions 504 or interlocking connectors. The electrode assembly then can be formed of a stack of loose, floating crowns that fit together via the extension portions and have substantially all of the outer circumference exposed to the surface of the lead 506. The segments can be bonded together and/or backfilled with a material such as silicone. The segments also can be attached by a connection means passing through a central region of each segment. Alternatively, the geometry can be created in a helical or other pattern out of a single component (e.g. a laser cut tube).

Overmolding

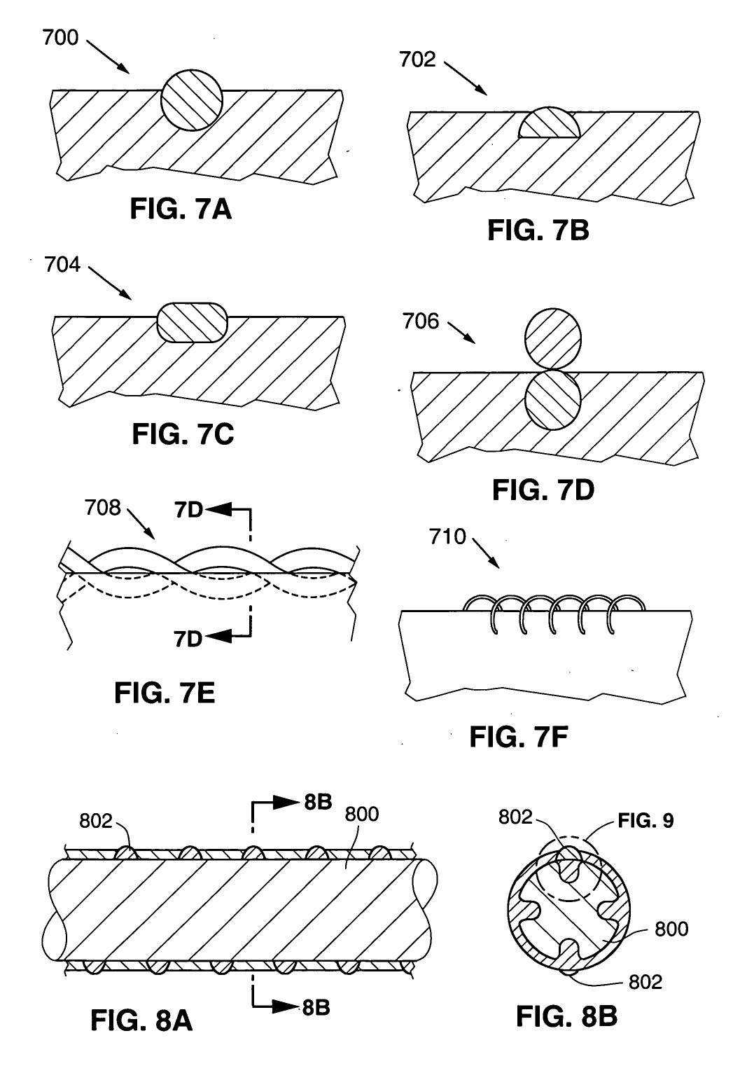

While the designs in FIGS. 2-5 can provide improvements for various applications, it can be desirable to utilize existing manufacturing steps while minimizing the amount of manufacturing changes necessary to provide an electrode that does not encourage fibrous ingrowth. As such, one can use an electrode assembly 600 in accordance with a fourth embodiment shown in FIGS. 6C and 6D. In this embodiment, a standard helical electrode 602 can be used, similar to those in the prior art. In this case, however, the helix is not positioned about the outermost circumference of the lead 604, but instead is wrapped about a smaller diameter section 603 (FIG. 6A) of the lead 604 such that the outer diameter of the helix is approximately equal to the outermost diameter of the lead. The electrode then can be overmolded with a material 606 such as silicone as shown in FIG. 6B. The overmold then can be selectively removed or ablated to expose the outer edge 608 of the electrode as shown in FIGS. 6C and 6D. Although the FIG. 6D embodiment uses an electrode having a circular cross-section (as also is the case in the FIG. 7A embodiment) FIGS. 7B-7D show examples 702 (D-shaped), 704 (rectangular or elliptical), 706 (twisted wire pair) of various alternative electrode cross-sections. FIG. 7E shows a side view of the electrode 706 of FIG. 7D illustrating that the electrode may comprise a twist 708 formed of a pair of conductors. FIG. 7F illustrates that the twist 708 of the FIG. 7D-7E embodiment may be replaced by a coil shaped electrode design. An overmold approach can be effective at smoothing the electrode surface and reducing the potential for ingrowth. Precision of the overmold process can be optimized by ablating with a laser or micro-blast device.

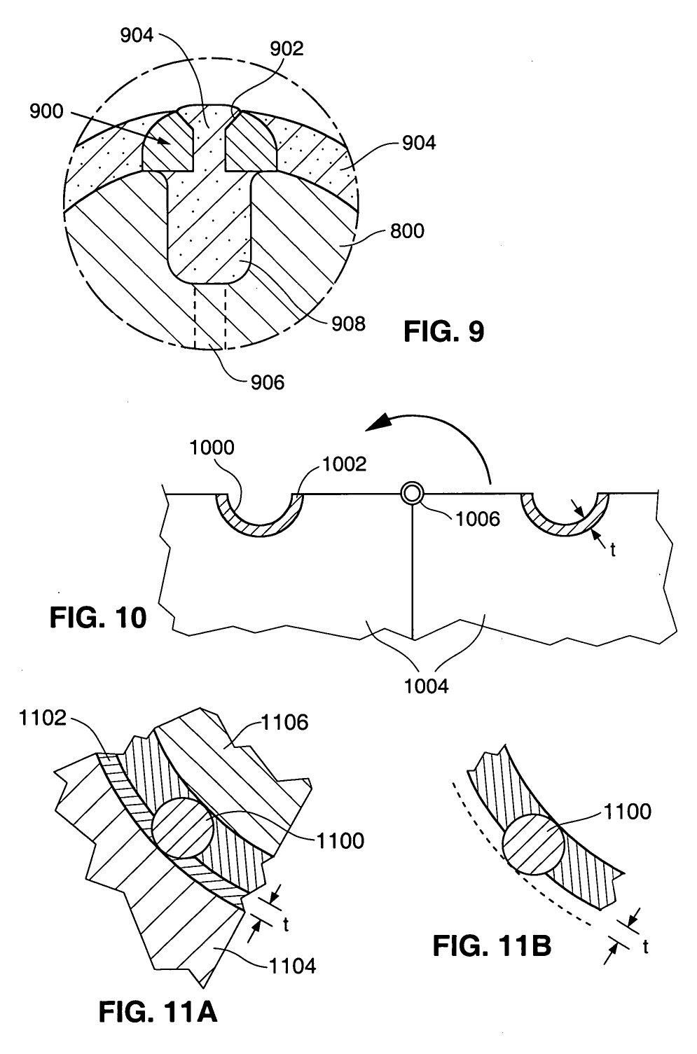

For some embodiments, particularly those in which the geometry make it difficult to provide a shut off on the electrode, the molding process may be facilitated through the use of an insert mold. In such cases, it can be useful to use a support mandrel 800 as shown in FIG. 8. A mandrel 800 can be used to support a molded electrode 802 or electrode sub-assembly, allowing the mold to shut-off against the electrode. The mandrel can be a lobed mandrel, as shown in the cross-section, which can be solid or drafted, for example. As shown in FIG. 9, the electrode 900 can optionally have holes 902 cross-drilled at specific intervals. Holes 902 can enhance the mechanical joint between the electrode and a molded elastomer 904 by permitting elastomer to flow through the holes 902 and to expand into the voids or recesses between the lobes of the mandrel, forming an internal web 908 as shown. To further facilitate a shutting-off of the electrode, a “soft” tool or insert can be utilized, such as a tool of Teflon®, silicone, polyurethane, or coated steel. The mandrel insert 800 shown in FIG. 9 further includes an optional gate location 906.

An alternative molding process uses a steel mold 1004 having a groove 1000 and a liner 1002 positioned in the groove 1000 as shown in FIG. 10. A hinge 1006 allows the parts of the mold tool 1004 to fold together to form the soft liner 1002 about the electrode and lead material, preferably with a mandrel 1106 positioned as shown in FIG. 11A, such that the molded material is molded between the mandrel and the liner. Such a liner can be temporary, disposable, or reusable. FIGS. 10 and 11A show that the relative height of the exposed electrode 1100 following molding is approximately equal to the thickness of the liner 1102, positioned between the tool 1104 and the mandrel 1106. Thus, as shown in FIG. 11B, once the mold is removed and the liner dissolved, a thickness of electrode 1100 is exposed that is approximately that of the removed liner.

Although certain electrode configurations have been described, a number of alternate electrode forms can be used, including but not limited to as ribbons, wire, mesh, metallic wool, and a doped elastomer (e.g. silicone doped w/medal particles or fibers). Alternative elastomers include but are not limited to porous/microporous silicone, such as can be created by filling silicone with a salt of a particular size, and then leaching the salt away with water, as well as by or drilling microholes with a laser (e.g. excimer). Other elastomers that can be used include hydrogels, tightly woven fabrics such as polyester, and ePTFE.

It should be recognized that a number of variations of the above-identified embodiments will be obvious to one of ordinary skill in the art in view of the foregoing description. Accordingly, the invention is not to be limited by those specific embodiments and methods of the present invention shown and described herein. Rather, the scope of the invention is to be defined by the following claims and their equivalents.

Any and all patents, patent applications and printed publications referred to above, including those relied upon herein for purposes of priority, are fully incorporated by reference.

What is claimed is:

1. A method of forming an electrode on an implantable lead, comprising the steps of:
encapsulating a plurality of electrode segments within an encapsulating material; and
removing a portion of the encapsulating material from each of the electrode segments to form an exposed electrode.

2. The method according to claim 1, wherein the encapsulating step includes encapsulating a helical coil, and wherein the plurality of electrode segments comprising windings of the helical coil.

3. The method according to claim 2, wherein the encapsulating step encapsulates the helical coil with a longitudinal axis of the helical coil positioned coaxially with a longitudinal axis of the lead.

4. The method according to claim 2, wherein the encapsulating step encapsulates the helical coil with a longitudinal axis of the helical coil laterally offset from a longitudinal axis of the lead.

5. The method according to claim 1, wherein the encapsulating step encapsulates segments having cross-sections selected from the group consisting of circular cross-sections, D-shaped cross sections, elliptical cross-sections, rectangular cross-sections, and twisted pair cross-sections.

6. The method according to claim 1, wherein at least one of the electrodes includes a channel therethrough, and wherein the encapsulating step includes causing encapsulating material to flow into the channel.

7. The method according to claim 1, wherein the encapsulating step includes the step of forming encapsulating material onto a mandrel.

8. The method according to claim 4, wherein the encapsulating step includes the step of forming encapsulating material onto a mandrel, and wherein the causing step causes encapsulating material to flow through the channel into a recess in the mandrel.

9. An implantable lead, comprising:
an elongate lead body; and
at least one electrode segment electrically coupled to the pulse generator, the electrode segment forming a substantially smooth edge with the pulse generator.

10. The implantable lead of claim 9, further including at least one electrical conductor coupled to the electrode segment, the conductor adapted for electrical communication with a pulse generator.

11. The implantable lead of claim 9, wherein the electrode segment is partially encapsulated by an encapsulating material.

12. The implantable lead of claim 11, wherein the electrode segment includes a curved exterior surface.

13. The implantable lead of claim 12, wherein the electrode segment has a cross-section selected from the group consisting of circular cross-sections, D-shaped cross sections, and elliptical cross-sections.

14. The implantable lead of claim 9, wherein the implantable lead includes a plurality of electrode segments.

15. The implantable lead of claim 9, wherein the implantable lead includes a helical electrode partially encapsulated in an encapsulating material.

16. The implantable lead of claim 15, wherein the helical electrode includes a plurality of regions exposed from the encapsulating material to form a plurality of electrode segments.

17. The implantable lead of claim 16, wherein the helical electrode has a longitudinal axis coaxial with a longitudinal axis of the lead body.

18. The implantable lead of claim 16, wherein the helical electrode has a longitudinal axis laterally offset from a longitudinal axis of the lead body.

19. The implantable lead of claim 9, wherein the implantable lead includes at least two conductors twisted around one another and partially encapsulated in an encapsulating material.

20. The implantable lead of claim 14, wherein the plurality of electrode segments comprise a plurality of spaced apart rings.

21. The implantable lead of claim 20, wherein the rings include longitudinally extending protrusions.

22. The implantable lead of claim 14, wherein the plurality of electrode segments comprise a bellows encapsulated in an encapsulating material, the bellows including outermost folds exposed from the encapsulating material to form the plurality of electrode segments.

23. The implantable lead of claim 20, further including flexible connectors coupling the rings.

24. The implantable lead of claim 9, wherein the lead body comprises an intravascular pulse generator.

25. The implantable lead of claim 9, wherein the lead body is proportioned for implantation in a right ventricle.

26. The implantable lead of claim 9, wherein an outer circumference of the at least one electrode segment is substantially equal to an outer circumference of the lead body.
